(19) United States
(12) Patent Application Publication
(10) Pub. No.: US 2006/0002997 A1
(43) Pub. Date: Jan. 5, 2006

(54) Nitrofurantoin controlled release dosage form

(76) Inventors: Puneet Shamar, Uttar Pradesh (IN); Pananchukunnath Manoj Kumar, New Delhi (IN); Vishnubhotla Nagaprasad, Kukatpally, Hyderabad (IN); Sunilendu Bhushan Roy, Mumbai, Maharashtra (IN); Rajiv Malik, Gangotri Enclave, Alknanda (IN)

Correspondence Address: RANBAXY INC., 600 COLLEGE ROAD EAST, SUITE 2100, PRINCETON, NJ, 08540, US

(21) Appl. No.: 10/525,534

(22) PCT Filed: Aug. 25, 2003
(86) PCT No.: PCT/IB03/03517

§ 371 Date: Feb. 23, 2005

(30) Foreign Application Priority Data
Aug. 23, 2002 (IN) 860/DEL/2002

(51) Int. Cl. A61K 9/48; A61K 9/24

(52) U.S. Cl. 424/451; 424/472

(57) ABSTRACT

The present invention generally relates to controlled release dosage forms which provide immediate release and sustained release of nitrofurantoin, and processes for their preparation. The controlled release dosage form includes a sustained release portion and an immediate release portion. The sustained release portion includes nitrofurantoin and one or more pH dependent hydrophilic polymers. The immediate release portion includes nitrofurantoin.

### FIELD OF THE INVENTION

The present invention generally relates to controlled release dosage forms which provide immediate release and sustained release of nitrofurantoin, and processes for their preparation.

### BACKGROUND OF THE INVENTION

Nitrofurantoin, chemically known as 1-[(5-nitrofurfurylidene) hydantoin, is a well known antibacterial agent for the treatment of urinary tract infections. Nitrofurantoin is a remarkably well-tolerated drug; however, it has side effects of nausea and emesis, which occasionally occur with after its oral administration. U.S. Pat. No. 3,401,221 discloses use the of macrocrystalline nitrofurantoin to reduce these side effects.

Further, U.S. Pat. No. 4,772,473 discloses a combination sustained release/immediate release capsule for oral administration of nitrofurantoin for minimizing side effects of nausea and emesis, and also for reducing the frequency of dosing from four times daily to twice daily. The immediate release layer described in the '473 patent includes macrocrystalline nitrofurantoin and the sustained release layer includes nitrofurantoin and a combination of polyvinylpyrrolidone and carboxyvinyl polymer as sustained release polymers.

### SUMMARY OF THE INVENTION

In one general aspect there is provided a controlled release dosage form that includes a sustained release portion and an immediate release portion. The sustained release portion includes nitrofurantoin and one or more pH dependent hydrophilic polymers. The immediate release portion includes nitrofurantoin.

Embodiments of the dosage form may include one or more of the following features. For example, the sustained release portion may further include one or more pH independent hydrophilic polymers. The sustained release portion may include two pH dependent hydrophilic polymers.

The pH dependent hydrophilic polymer may include one or more of cross-linked acrylic acid polymers and methacrylic acid derivatives. The cross-linked acrylic acid polymers may be carboxyvinyl polymers. The carboxyvinyl polymer may be one or more of Carbopol® 974P, Carbopol® 971P, and Carbopol® 934P or a combination of Carbopol® 974P and Carbopol® 971P. The methacrylic acid derivative may be one or more of Eudragit® L and Eudragit® S.

The one or more pH independent hydrophilic polymers may be cellulose ethers. The cellulose ethers may be one or more of hydroxypropyl methylcellulose and hydroxypropyl cellulose. The cellulose ethers may be one or more low viscosity hydroxypropyl celluloses having a molecular weight of about 80,000-100,000.

The nitrofurantoin may be macrocrystalline nitrofurantoin. The nitrofurantoin may have a particle size distribution with D90<250 mm.

The sustained release portion may further include one or more pharmaceutically acceptable excipients. The sustained release portion may be one or more of powder, granules, compact or tablet and in particular may be a tablet. The immediate release portion may be one or more of powder or granules and in particular may be powder.

The dosage form may be a capsule. The dosage form may have a dissolution profile in which approximately eight percent to approximately twenty percent of the nitrofurantoin in the dosage form is released within one hour in an approximately 0.01N HCl solution and the majority of the remaining nitrofurantoin in the dosage form is released over seven hours in a phosphate buffer having a pH of approximately 7.5, the dissolution profile being measured using a USP apparatus 2 at a paddle speed of approximately 100 rpm and a temperature of approximately 37° C.

In another general aspect there is provided a process for the preparation of a controlled release dosage form that includes a sustained release portion and an immediate release portion. The process includes preparing the sustained release portion in a process that includes blending nitrofurantoin with one or more pH dependent hydrophilic polymers; preparing the immediate release portion by providing nitrofurantoin; and filling the sustained release portion and the immediate release portion into the dosage form.

Embodiments of the process may include one or more of the following features. For example, preparing the sustained release portion may further include mixing and blending the nitrofurantoin with one or more pharmaceutically acceptable excipients. The sustained release portion may be powder, granules, compact or tablet.

The process may further include blending the sustained release portion with one or more pH independent hydrophilic polymers. The pH independent hydrophilic polymer may be one or more cellulose ethers. The one or more cellulose ethers may be one or more of hydroxypropyl methylcellulose and hydroxypropyl cellulose. The hydroxypropyl cellulose may have a low viscosity and a molecular weight of about 80,000-100,000.

The sustained release portion may further include two pH dependent hydrophilic polymers. The pH dependent hydrophilic polymer may be one or more of cross-linked acrylic acid polymers and methacrylic acid derivatives. The cross-linked acrylic acid polymers may be one or more carboxyvinyl polymers. The carboxyvinyl polymer may be one or more of Carbopol® 974P, Carbopol® 971P, and Carbopol® 934P or a combination of Carbopol® 974P and Carbopol® 971P. The one or more methacrylic acid derivatives may be one or both of Eudragit® L and Eudragit® S.

Preparing the immediate release portion may further include blending the nitrofurantoin with one or more pharmaceutically acceptable excipients. The immediate release portion may be powder or granule. The immediate release portion may be filled into the dosage form before the sustained release portion is filled into the dosage form or the immediate release portion may be filled into the dosage form after the sustained release portion is filled into the dosage form.

The nitrofurantoin in the immediate release portion may be macrocrystalline nitrofurantoin. The nitrofurantoin in the immediate release portion may have a particle size distribution with D90<250 mm.

The dosage form may be a capsule. The dosage form may have a dissolution profile in which approximately eight percent to approximately twenty percent of the nitrofurantoin in the dosage form is released within one hour in an approximately 0.01N HCl solution and the majority of the remaining nitrofurantoin in the dosage form is released over seven hours in a phosphate buffer having a pH of approximately 7.5, the dissolution profile being measured using a USP apparatus 2 at a paddle speed of approximately 100 rpm and a temperature of approximately 37° C.

In another general aspect, there is provided a method of treating a urinary tract infection that includes administering a controlled release dosage form. The dosage form includes a sustained release portion that includes nitrofurantoin and one or more pH dependent hydrophilic polymers and an immediate release portion that includes nitrofurantoin.

Embodiments of the method may include one or more of the following features including those described above. For example, the dosage form may have a dissolution profile in which approximately eight percent to approximately twenty percent of the nitrofurantoin in the dosage form is released within one hour in an approximately 0.01N HCl solution and the majority of the remaining nitrofurantoin in the dosage form is released over seven hours in a phosphate buffer having a pH of approximately 7.5, the dissolution profile being measured using a USP apparatus 2 at a paddle speed of approximately 100 rpm and a temperature of approximately 37° C.

The details of one or more embodiments of the inventions are set forth in the description below. Other features, objects, and advantages of the inventions will be apparent from the description and the claims.

### DETAILED DESCRIPTION OF THE INVENTION

We have now discovered a novel combination of sustained release polymers that provides excellent sustained release properties to nitrofurantoin and maintains the therapeutic level of nitrofurantoin for more than twelve hours. The dosage form provides an immediate release portion and an extended or sustained release portion. The immediate release portion allows for rapid absorption of nitrofurantoin to quickly achieve therapeutic plasma levels. The sustained release portion maintains the achieved therapeutic levels of nitrofurantoin for a prolonged duration.

The nitrofurantoin sustained release portion may include one or more pH dependent polymers. When one pH dependent hydrophilic polymer is used, it is selected such that it produces a viscous gel and provides a near zero order release profile throughout the gastrointestinal tract. However, when more than one pH dependent hydrophilic polymer is employed, these are selected in such a way that at least one polymer provides a semi-enteric release profile, i.e., slow release in the stomach and immediate release in the intestine (above pH 6), and the other polymer provides slow and linear release throughout the intestinal tract.

The sustained release portion also may include a pH independent hydrophilic polymer. The addition of pH independent hydrophilic polymer provides cohesiveness to the mass so that the compact or tablet maintains its structure and integrity as it traverses the gastrointestinal tract.

Such a combination of polymers gels and/or swells in the gastric fluid to form a viscous matrix from which only a small amount of nitrofurantoin is released via diffusion. However, in the neutral and alkaline pH of the small intestine, the pH dependent hydrophilic polymer fully hydrates and slowly erodes to release the drug. The concentration and ratio of the pH dependent hydrophilic polymers is optimized in such a way that the desired release profile is obtained in the intestine. The pH dependent hydrophilic polymer(s) may be used in concentrations of about 2-20%, and the pH independent hydrophilic polymer(s) may be used in concentrations of about 0.01-15%.

As used herein, the term “Nitrofurantoin” includes nitrofurantoin, its pharmaceutically acceptable salts and hydrates. “Macrocrystalline nitrofurantoin” refers to particulate crystalline nitrofurantoin, for example, as described in the United States Pharmacopoeia. The nitrofurantoin as used in the sustained release portion is a micronized nitrofurantoin monohydrate having a particle size distribution with D_{90<250} μm. Reduction in particle size provides for greater surface area and hence better bioavailability. This also helps in uniform mixing of the drug with polymers and other excipients, both of which may have a similar particle size distribution.

The pH dependent hydrophilic polymer may be selected from crosslinked acrylic acid based polymers and methacrylic acid polymers. The crosslinked acrylic acid polymers include carboxyvinyl polymers commercially available under the trade name “carbopol” from Noveon Inc. Company, Cleveland, Ohio, USA. Particularly suitable are Carbopol® 974P, Carbopol® 971P and Carbopol® 934P. Both Carbopol® 974P and Carbopol® 934P are highly crosslinked, have similar viscosity profiles, but have different release patterns. Instead of the near zero order release profiles throughout the gastrointestinal tract observed in systems formulated with Carbopol® 934P, Carbopol® 974P provides a semi-enteric release profile. Carbopol® 971P is lightly cross-linked and provides a more linear and slow release. Similarly, methacrylic acid copolymers commercially available as Eudragit® by Rohm, Germany may be used. However use of Eudragit® L and S® is particularly suitable. The pH dependent hydrophilic polymers control the sustained release characteristics of the nitrofurantoin.

The pH independent hydrophilic polymer may be selected from cellulose ethers, such as hydroxypropyl methylcellulose and hydroxypropyl cellulose commercially available from Dow Chemicals, USA and M/s Nisso, Japan under the trade names Methocel® and HPC. Hydroxypropyl cellulose includes low viscosity grade polymers having molecular weights of about 80,000-100,000. Hydroxypropyl methylcellulose includes those having a viscosity of about 5-100 cps.

Besides the above polymers, the sustained release portion may also contain other pharmaceutically acceptable excipients such as diluents, binders, stabilizers, antioxidants, preservatives, wetting agents, lubricants, glidants and colors.

Suitable binders may include one or more of methyl cellulose, hydroxypropyl cellulose, hydroxypropyl methylcellulose, gelatin, gum arabic, ethyl cellulose, polyvinyl alcohol, pullulan, pregelatinized starch, agar, tragacanth, sodium alginate, propylene glycol, and the like. Suitable diluents may include one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, sugar confectioners and the like. Suitable lubricants and glidants may include one or more of colloidal anhydrous silica, stearic acid, sodium stearyl fumarate, magnesium stearate, calcium stearate, talc, hydrogenated caster oil, sucrose esters of fatty acid, microcrystalline wax, yellow beeswax, white beeswax and the like. The colors may be selected from any FDA approved colors for internal use. The formulation may optionally be coated.

The immediate release portion of the dosage form also may contain diluents, binders, disintegrants, and lubricants in addition to the nitrofurantoin. Suitable diluents, binders, disintegrants, and lubricants include those described above for sustained release portion.

The dosage form may be in tablet or capsule form, however, the capsule form is particularly suitable.

The immediate release portion of the dosage form is prepared by blending macrocrystalline nitrofurantoin with one or more pharmaceutically acceptable excipients and filing into a hard gelatin capsule. As the immediate release portion provides an immediate onset of action, the use of macrocrystalline nitrofurantoin is preferred due to the lower incidence of nausea and emesis. To maintain the crystal structure of macrocrystalline nitrofurantoin, compression is normally avoided as compression may break the crystals and reduce the particle size. Therefore, the immediate release portion is preferably filled with the macrocrystalline nitrofurantoin as such or in granular form.

The sustained release portion is prepared by mixing micronized nitrofurantoin monohydrate with sustained release polymers and other pharmaceutically acceptable excipients. The blend can be filled into a hard gelatin capsule as such, as granules, or as a loosely formed compact or tablet. The order in which the sustained release and immediate release mixtures are filled into the capsule shell can be varied, but preferably the two portions should form separate layers. The following examples further exemplify the inventions and are not intended to limit the scope of the inventions.

### EXAMPLE 1

| Immediate release portion | mg per capsule |
|---|---|
| Nitrofurantoin macrocrystals | 25 |
| Lactose | 126.0 |
| Starch | 122.50 |
| Magnesium stearate | 1.50 |
| Sustained Release portion | |
| Nitrofurantoin monohydrate (eq. | 75.00 |
| to Nitrofurantoin) | |
| Carbopol ® 971P | 1.70 |
| Carbopol ® 974P | 3.45 |
| Hydroxypropylcellulose -L | 2.50 |
| Talc | 2.20 |
| Aerosil -200 | 1.10 |
| Compressible sugar | 17.89 |
| Magnesium stearate | 1.0 |

Process

(Immediate Release Portion)
1. Nitrofurantoin, lactose and starch were sifted through a suitable mesh and mixed to form a blend.
2. Magnesium stearate was sifted through a suitable mesh, added to the above blend, and mixed well.
3. The final blend of step (2) was filled into a hard gelatin capsule of the appropriate size.

(Sustained Release Portion)
1. Nitrofurantoin and aerosil were sifted together through a suitable mesh, followed by sifting of Carbopol® 971P, Carbopol® 974P, hydroxypropylcellulose-L, sugar and talc through a suitable mesh. The ingredients then were mixed thoroughly to form a blend.
2. The blend was compacted and sized through a suitable mesh.
3. The compacted and sized blend was lubricated with magnesium stearate and compressed to form a tablet.
4. The resulting tablet was filled into the hard gelatin capsule into which the immediate release portion had been filled.

### EXAMPLE-2

| Immediate release portion | mg per capsule |
|---|---|
| Nitrofurantoin macrocrystals | 25 |
| Lactose | 126.0 |
| Starch | 122.50 |
| Magnesium stearate | 1.50 |
| Sustained Release portion | |
| Nitrofurantoin monohydrate (eq. | 75.00 |
| to Nitrofurantoin) | |
| Carbopol ® 934P | 4.00 |
| Hydroxypropyl methyl cellulose | 8.00 |
| Talc | 1.0 |
| Corn ressible sugar | 15.14 |
| Sodium stearyl fumarate | 1.70 |

Process

(Immediate Release Portion)
1. Nitrofurantoin, lactose and starch were sifted through a suitable mesh and mixed to form a blend.
2. Magnesium stearate was sifted through a suitable mesh, added to the above blend, and mixed well.
3. The final blend was filled into a hard gelatin capsule of appropriate size.
   (Sustained Release Portion)

Nitrofurantoin, Carbopol® 934P, hydroxypropyl methylcellulose, sugar and talc were sifted through a suitable mesh. All ingredients were mixed thoroughly to form a blend.
1. The blend was compacted and sized through suitable mesh.
2. The compacted and sized blend was lubricated with sodium stearyl fumarate and compressed.
3. The resulting tablet was filled into the hard gelatin capsule into which the immediate release portion had been filled.

Samples of the capsules prepared according to Examples 1 and 2 were subjected to dissolution testing using USP apparatus 2, paddle speed 100 rpm, temperature 37° C., in simulated gastric fluid (0.01N HCl) for 1 hour, followed by a further 7 hours in a phosphate buffer (pH 7.5). The samples were taken from the dissolution medium at different time intervals and analyzed for nitrofurantoin. The immediate or rapid release performance and the sustained release performance of the capsules of Examples 1 and 2 are shown in Tables 1 and 2, respectively. Tables 1 and 2 show that at in one hour least ten percent of the nitrofurantoin is released in the simulated gastric fluid, representing release in the stomach, and the remaining nitrofurantoin is released over seven hours in the phosphate buffer, which represents release in the intestine.

TABLE 1
Dissolution of Nitrofurantoin capsules of Example 1

| | Cumulative % of Nitrofurantoin released | |
|---|---|---|
| Time | Dissolution media | Dissolution media |
| (hours) | 0.01N HCl | pH 7.5 Phosphate buffer |
| 1 | 11 | — |
| 2 | | 43 |
| 3 | | 60 |
| 4 | | 76 |
| 5 | | 87 |
| 6 | | 91 |
| 7 | | 98 |
| 8 | | 104 |

TABLE 2
Dissolution of Nitrofurantoin capsules of Example 2

| | Cumulative % of Nitrofurantoin released | |
|---|---|---|
| Time | Dissolution media | Dissolution media |
| (hours) | 0.01N HCl | pH 7.5 Phosphate buffer |
| 1 | 12 | — |
| 2 | | 48 |
| 3 | | 60 |
| 4 | | 70 |
| 5 | | 76 |
| 6 | | 80 |
| 7 | | 90 |
| 8 | | 98 |

While several particular forms of the inventions have been described, it will be apparent that various modifications and combinations of the inventions detailed in the text can be made without departing from the spirit and scope of the inventions. For example, although the examples above are directed to application of the inventive concepts described herein to nitrofurantoin as the active pharmaceutical ingredient, these concepts can be applied to other active pharmaceutical ingredients, such antidiabetics, antineoplastic agents, antihypertensives, psychopharmacological agents, cardiovascular agents, platelet aggregation inhibitors, analgesics, antimicrobials, diuretics, spasmolytics, and the like. Moreover, one or more additional active pharmaceutical ingredients can be used in either or both of the sustained release portion and the immediate release portion. Finally, it is contemplated that any single feature or any combination of optional features of the inventive variations described herein may be specifically excluded from the claimed inventions and be so described as a negative limitation. Accordingly, it is not intended that the inventions be limited, except as by the appended claims.

1. A controlled release dosage form comprising:
a sustained release portion comprising nitrofurantoin and one or more pH dependent hydrophilic polymers; and
an immediate release portion comprising nitrofurantoin.

2. The dosage form according to claim 1 wherein the sustained release portion further comprises one or more pH independent hydrophilic polymers.

3. The dosage form according to claim 1 wherein the sustained release portion comprises two pH dependent hydrophilic polymers.

4. The dosage form according to claim 1 wherein the pH dependent hydrophilic polymer comprises one or more of cross-linked acrylic acid polymers and methacrylic acid derivatives.

5. The dosage form according to claim 4 wherein the cross-linked acrylic acid polymers comprise carboxyvinyl polymers.

6. The dosage form according to claim 5 wherein the carboxyvinyl polymer comprises one or more of Carbopol® 974P, Carbopol® 971P, and Carbopol® 934P.

7. The dosage form according to claim 5 wherein the carboxyvinyl polymer comprises a combination of Carbopol® 974P and Carbopol® 971 P.

8. The dosage form according to claim 4 wherein the methacrylic acid derivative comprises one or more of Eudragit® L and Eudragit® S.

9. The dosage form according to claim 2 wherein the one or more pH independent hydrophilic polymers comprise cellulose ether.

10. The dosage form according to claim 9 wherein the cellulose ether comprises one or more of hydroxypropyl methylcellulose and hydroxypropyl cellulose.

11. The dosage form according to claim 10 wherein the cellulose ether comprises one or more low viscosity hydroxypropyl celluloses having a molecular weight of about 80,000-100,000.

12. The dosage form according to claim 1 wherein the nitrofurantoin comprises macrocrystalline nitrofurantoin.

13-18. (canceled)

19. The dosage form of claim 1 wherein the dosage form has a dissolution profile in which approximately eight percent to approximately twenty percent of the nitrofurantoin in the dosage form is released within one hour in an approximately 0.01N HCl solution and the majority of the remaining nitrofurantoin in the dosage form is released over seven hours in a phosphate buffer having a pH of approximately 7.5, the dissolution profile being measured using a USP apparatus 2 at a paddle speed of approximately 100 rpm and a temperature of approximately 37° C.

20. A process for the preparation of a controlled release dosage form comprising a sustained release portion and an immediate release portion, the process comprising:
preparing the sustained release portion in a process comprising blending nitrofurantoin with one or more pH dependent hydrophilic polymers;
preparing the immediate release portion by providing nitrofurantoin; and filling the sustained release portion and the immediate release portion into the dosage form.

21. The process of claim 20 wherein preparing the sustained release portion further comprises mixing and blending the nitrofurantoin with one or more pharmaceutically acceptable excipients.

22. The process of claim 20 wherein preparing the immediate release portion further comprises blending the nitrofurantoin with one or more pharmaceutically acceptable excipients.

23. (canceled)

24. The process of claim 20 wherein the immediate release portion is filled into the dosage form before the sustained release portion is filled into the dosage form.

25. The process of claim 20 wherein the immediate release portion is filled into the dosage form after the sustained release portion is filled into the dosage form.

26. The process of claim 20 wherein the nitrofurantoin in the immediate release portion comprises macrocrystalline nitrofurantoin.

27. The process of claim 20 wherein the nitrofurantoin in the immediate release portion has a particle size distribution with D_{90<250} μm.

28. The process of claim 20 further comprising blending the sustained release portion with one or more pH independent hydrophilic polymers.

29-40. (canceled)

41. The process of claim 20 wherein the dosage form has a dissolution profile in which approximately eight percent to approximately twenty percent of the nitrofurantoin in the dosage form is released within one hour in an approximately 0.01N HCl solution and the majority of the remaining nitrofurantoin in the dosage form is released over seven hours in a phosphate buffer having a pH of approximately 7.5, the dissolution profile being measured using a USP apparatus 2 at a paddle speed of approximately 100 rpm and a temperature of approximately 37° C.

42. A method of treating a urinary tract infection comprising administering a controlled release dosage form, the dosage form comprising:
a sustained release portion comprising nitrofurantoin and one or more pH dependent hydrophilic polymers; and
an immediate release portion comprising nitrofurantoin.

43. The method of claim 42 wherein the dosage form has a dissolution profile in which approximately eight percent to approximately twenty percent of the nitrofurantoin in the dosage form is released within one hour in an approximately 0.01N HCl solution and the majority of the remaining nitrofurantoin in the dosage form is released over seven hours in a phosphate buffer having a pH of approximately 7.5, the dissolution profile being measured using a USP apparatus 2 at a paddle speed of approximately 100 rpm and a temperature of approximately 37° C.
